(19) United States
(12) Patent Application Publication
(10) Pub. No.: US 2026/0250661 A1
(43) Pub. Date: Aug. 27, 2026

(54) LIBRARY OF PROXIMITY PROBES AND METHOD OF USE

(71) Applicant: Olink Proteomics AB, Uppsala (SE)

(72) Inventors: Erika ASSARSSON, Uppsala (SE); Martin LUNDBERG, Uppsala (SE); Niklas NORDBERG, Storvreta (SE); Sandra OHLSSON, Uppsala (SE); Pascal PUCHOLT, Uppsala (SE)

(21) Appl. No.: 19/144,069

(22) PCT Filed: Nov. 10, 2023
(86) PCT No.: PCT/EP2023/081444

§ 371 Date: Jun. 27, 2025

(30) Foreign Application Priority Data
Nov. 11, 2022 (EP) 22206994.0

(51) Int. Cl. C12N 15/10; C40B 40/06; G16B 25/20

(52) U.S. Cl. CPC C12N 15/1055; C40B 40/06; G16B 25/20

(57) ABSTRACT

The present invention relates to a library comprising a plurality of detection probes suitable for detection of a set of analytes of interest, each detection probe comprising an analyte-specific binding domain and a nucleic acid moiety, the nucleic acid moiety of a detection probe being capable of generating a reporter nucleic acid molecule comprising an identification sequence identifying the respective analyte of interest, said library being wherein the library comprises, for one or more analytes of interest, a set of two or more detection probes wherein each detection probe within the set is capable of generating a reporter nucleic acid molecule comprising an identification sequence that differ from the identification sequences generated by the other members of the same set. The invention further relates to a method for composing a panel with use of the library, as well as a computer program implementing said method.

### FIELD OF THE INVENTION

The present invention relates to tools for biotechnological research, and in particular products and methods for quick and efficient development of custom-made multiplex panels of assays for analytes of interests. The invention finds particular use in development of flexible panels for use in the research field of proteomics.

### BACKGROUND

Modern proteomics methods require the ability to detect a large number of different proteins (or protein complexes) in a small sample volume. To achieve this, multiplex analysis must be performed. Common methods by which multiplex detection of proteins in a sample may be achieved include proximity extension assays (PEA) and proximity ligation assays (PLA). PEA and PLA are described in WO 01/61037; PEA is further described in WO 03/044231, WO 2004/094456, WO 2005/123963, WO 2006/137932, WO 2013/113699, WO 2021/191442, WO 2021/191448, WO 2021/191449, WO 2022/191450, and WO 2022/112300; Assarsson et al., PLOS 1, 2014, 9, 4, e95192; Lundberg et al., Molecular & Cellular Proteomics 10:10.1074/mcp.M110.004978, 1-10, 2011; and Wik et al., 2021, Mol Cell Proteomics 20, 100168, all incorporated herein by reference in their entirety.

When, as is common, the proteins of interest are present in a wide concentration range, this presents a challenge, since the signal from proteins of high concentration may overwhelm the signal from proteins of low concentration, resulting in a failure to detect proteins present at lower concentrations.

PEA and PLA are proximity assays, which rely on the principle of “proximity probing”. In these methods, an analyte is detected by the binding of multiple (i.e. two or more, generally two or three) probes, which when brought into proximity by binding to the analyte (hence “proximity probes”) allow a signal to be generated. Typically, at least one of the proximity probes comprises a nucleic acid domain (or moiety) linked to the analyte-binding domain (or moiety) of the probe, and generation of the signal involves an interaction between the nucleic acid moieties and/or a further functional moiety which is carried by the other probe(s). Thus, signal generation is dependent on an interaction between the probes (more particularly between the nucleic acid or other functional moieties/domains carried by them) and hence only occurs when the necessary probes have bound to the analyte, thereby lending improved specificity to the detection system.

In PEA, nucleic acid moieties linked to the analyte-binding domains of a probe pair hybridise to one another when the probes are in close proximity (i.e. when bound to the same target molecule, or to target molecules which are in close proximity, for example in a complex, interaction, or aggregate, or when two molecules are closely co-located), and are then extended using a nucleic acid polymerase. The extension product forms a reporter nucleic acid, detection of which demonstrates the presence of a particular analyte (the analyte bound by the relevant probe pair) in a sample of interest. In PLA, nucleic acid moieties linked to the analyte-binding domains of a probe pair come into proximity when the probes of the probe pair bind their target, and may be ligated together, or alternatively they may together template the ligation of separately added oligonucleotides which are able to hybridise to the nucleic acid domains when they are in proximity. The ligation product is then amplified, acting as a reporter nucleic acid. Multiplex analyte detection using PEA or PLA may be achieved by including a unique barcode sequence in the nucleic acid moiety of each probe. A reporter nucleic acid molecule corresponding to a particular analyte may be identified by the barcode sequences it contains. The methods of the present invention find particular utility in multiplex PEA and PLA methods.

Panels of proximity assays, as described above, are commercially available from Olink Proteomics AB (Uppsala, Sweden) under the trademark Olink® Target, Olink® Focus, and Olink® Explore. These are fixed panels of up to 92 assays (Olink® Target and Focus) or up to ~3,000 assays split over eight different panels (Olink® Explore). Each panel generally includes assays for proteins that have known functions within certain biological or physiological areas, pathways or organs in the body, such as inflammation, organ-specific proteins, cardiovascular, neurology etc.

Enroth et al., Communications Biology 2(1), 2019, pages 1-12 (DOI: 10.1038/s 42003-019-0464-9) describes the use of Olink Panels, and custom-designed panels, in PEA assays to identify a novel high accuracy plasma protein biomarker signature for ovarian cancer.

Currently commercially available immunoassays allowing users to combine individual assays into a multiplex assay have a combinability of 80-90%. That is, 10-20% of the theoretically possible combined multiplex panels are not available due to technical constraints. Furthermore, even if the assays are combinable, they may need to be run in separate reaction containers.

### SUMMARY

The invention is as set out in the appended claims.

### BRIEF DESCRIPTION OF THE DRAWINGS

FIG. 1 is a visualization of a distribution of identification sequences over a set of detection probes.

### DEFINITION OF TERMS AND ABBREVIATIONS

All terms and abbreviations used in the present specification shall be construed to have the meaning normally given to them in the relevant art, unless another meaning is clearly intended. For the sake of clarity, a few terms and abbreviations are defined below.

A “library” is a collection of items and/or sets of items. A library is generally ordered and structured so that all items and sets of items are separately retrievable.

“Readout” is intended to refer to the process of quantifying the amount of reporter molecules with the respective unique identification sequences and correlating these amounts to the amounts of the respective analytes of interest in the analyzed sample. Accordingly, a readout can be seen as a step of detecting the signal in the assay, or more particularly the reporter molecules, in a quantitative manner.

### DETAILED DESCRIPTION

The present invention relates to means and methods for flexible composition of multiplex biological assays based on generation of reporter nucleic acids wherein the amount of a specific reporter nucleic acid molecule corresponds to the amount of a specific analyte of interest in the sample. Such assays are known in the art and include i.a. PLA and PEA as described above. However, there is a need for means and methods for quick, easy, and cheap composition of new panels with new combinations of assays.

Multiplexing of biological assays, such as proximity assays, means performing a plurality of assays in parallel and, preferably, in the same reaction container, e.g. a test tube or a well in a microtiter plate. Multiplexing thus has the potential to massively increase throughput of samples and reduce foot-print of the necessary equipment.

Combining a plurality of biological assays on the same sample and in the same container, may cause interference between assays in many ways and is thus not straightforward. One particular interference that need to be resolved for multiplexed assays based on correlating the amount of reporter nucleic acid molecules comprising identification sequences to the analytes of interest is to avoid overlap of identification sequences, so that no identification sequence is connected to more than one analyte of interest. That is, that each identification sequence is unique to an analyte of interest, within any given panel of assays.

The identification sequence may e.g. be a unique sequence (usually termed a “barcode sequence” or simply “barcode”) that is detected in a sequence-specific manner, for example, sequenced for identification in the readout step, or which provides a specific binding (hybridization) site for a probe or primer used in the detection, e.g., a unique primer binding site that can be used for readout using quantitative PCR (qPCR).

A common solution to the above described overlap problem is to provide fixed panels of assays, where any overlap problems are resolved during development of the specific fixed panels prior to commercial launch. Fixed multiplex assay panels of varying size and assay content provide an ideal broad screening-to-targeted discovery solution for many explorative applications and research questions.

There may, however, be situations where no existing fixed panel is suitable for the research question at hand, and a user wishes to design a panel of assays for a particular set of analytes of interest. While it is possible in each such case to assign unique identification sequences to each detection probe to avoid overlap within the newly designed panel, this takes time both in the design process and physical manufacturing. It would be advantageous to have a library of ready-made detection probes with pre-assigned and unique identification sequences available.

It may also be desirable to limit the available pool of identification sequences. For instance, when the identification sequences comprise unique primer binding sites, it is desirable to limit the number of unique primer binding sites in order to be able to use the same set of readout primers for any panel, regardless of the selected assay content.

The present invention aims to provide a library of detection probes that facilitate flexible design of panels of assays that can be quickly produced with ready-made detection probes, optionally with a limited pool of identification sequences.

The present invention further aims to facilitate fast and easy compilation and manufacture of a custom-made, multiplexed, panel of biological assays based on connecting nucleic acid molecules capable of generating reporter nucleic acid molecules comprising identification sequences to the analytes of interest, wherein each of the analytes of interest are assayed based on the abundance of reporter nucleic acid molecules comprising an identification sequence that is unique to the specific analyte of interest.

To this aim the present invention, in one aspect, provides a library comprising a plurality of detection probes for detection of a plurality of analytes of interest, each detection probe comprising an analyte-specific binding domain and a nucleic acid moiety, the nucleic acid moiety of a detection probe being capable of generating a reporter nucleic acid molecule comprising an identification sequence identifying the respective analyte of interest, said library comprising, for one or more analytes of interest, a set of two or more detection probes wherein each detection probe within the set is capable of generating a reporter nucleic acid molecule comprising an identification sequence that differ from the identification sequences generated by the other members of the same set.

By appropriately adjusting the number of members for each set of probes, the library provides a convenient means for facilitating combinations of detection probes for any, or almost any, subset of analytes of interest, wherein all detection probes within the combination have unique identification sequences.

In other words, the provision of the library according to the invention makes it possible to quickly select a panel of assays for detection of a number of analytes of interest, by selecting detection probes from the library for inclusion in the panel so that each selected probe is capable of generating a reporter nucleic acid molecule having an identification sequence that is unique within the selected panel. Methods for such selection are included within the present invention.

Thus, for a given analyte, the library may comprise a set of detection probes which can give rise to reporter molecules with different identification (ID) sequences (i.e. different detection probes for a particular analyte), from which a user can select a detection probe for inclusion in a panel of assays. It is not required that for each and every analyte a set of different detection probes is included, but rather that at least for some analytes, or for a number or plurality of analytes, a set of such different detection probes is included. Accordingly, the library can be seen to comprise a number, or a plurality of, sets of detection probes, wherein a set of detection probes comprises two or more detection probes for detection of a given analyte (i.e. the same analyte). In other words, the library comprises a separate set of detection probes for one or more analytes. A set of detection probes is specific for a particular analyte (i.e. the same analyte is detected by the set), but within a set the reporter nucleic acid molecule generated by a detection probe is different (or more particularly comprises a different ID sequence).

A detection probe comprises, as discussed herein, at least one analyte-specific binding domain and at least one nucleic acid moiety. The analyte-specific binding domain and the nucleic acid moiety may be covalently or non-covalently bonded to each other. A detection probe may further comprise one or more components or parts. Thus, it may be a single probe comprising one analyte-specific binding domain and one nucleic acid moiety, or a probe provided in two or more parts, or a group or suite of probes for use together (i.e. in combination) to detect the analyte. Thus, the detection probe may comprise, or be provided by, proximity probes. Proximity probes are typically used in pairs, but in certain embodiments more than two proximity probes may be used together for detection of a given analyte, for example, 3 proximity probes. Proximity probes of a pair or more are referred to as matched, or cognate for one another.

The analyte of interest may be any analyte it is desired to detect. In an embodiment, the analyte is a protein. However, it may be any biological or chemical entity it desired to detect. As indicated above, proximity probes are used in the art to detect a wide variety of analytes, and these may include interactions and complexes etc. Thus, the proximity probes of a pair (or more) may each bind to the same target molecule (but at different sites, so that the individual proximity probes may each bind to their respective target binding sites at the same time (i.e. simultaneously), or to different molecules (e.g. where the target analyte is an interaction, and each proximity probe binds to a different member of the interaction, or where a post-translational modification of a given protein is being detected). Indeed, the target analyte may be the co-localization of two molecules in close proximity.

The reporter nucleic acid molecule may be generated from the nucleic acid moiety(ies) of the detection probe in a variety of ways. This may include amplification, extension, ligation and/or cleavage. In the context of proximity probes, the possibilities are discussed in the documents cited above. The identification sequence, or a part thereof, may be included in the detection probe (or one or more parts or components thereof), or may be generated by the generation of the reporter nucleic acid molecule.

As is known in the art, the analyte-specific binding domain of a detection probe may be any entity capable of binding specifically to a target analyte (or part thereof), and being coupled to a nucleic acid moiety. That the binding domain is “specific” to a certain analyte means, as is known to the skilled person, that it recognizes the analyte with low cross-reactivity (off-target binding) with other potentially present analytes, within the relevant application and experimental context. A framework for determining specificity for binders have been established by an International Working Group for Antibody Validation (Uhlén et al. Nat Methods, 2016 October; 13(10), 823-827, incorporated herein by reference).

Typically, the analyte-specific binding domain may be a protein, for example, an antibody, or an antigen-binding part thereof, including, but not limited to, monoclonal, recombinant monoclonal, and polyclonal antibodies and antigen-binding antibody derivatives and fragments. However, the analyte-specific binding domain may be of any nature, including lectins, soluble cell surface receptors, combinatorially derived proteins from phage display or ribosome display, peptides, carbohydrates, molecularly imprinted polymers (MIPs), nucleic acids, such as an aptamer or a nucleic acid molecule comprising the complementary sequence for a target nucleic acid, or combinations thereof.

Reagents useful as analyte-specific binding domains are commercially available from a number of manufacturers that offer off-the-shelf reagents or develop new binding reagents for specific analytes and specific needs. Such manufacturers include, among others, Thermo Fisher Scientific (Boston, MA, USA), Abcam (Cambridge, United Kingdom), Bio-Techne (Minneapolis, MN, USA), Proteogenix (Schiltigheim, France), Sino Biological (Beijing, China), Agrisera (Vännäs, Sweden), Novaptech (Pessac, France), Aptamer Group (York, United Kingdom). Reagents useful as analyte-specific binding domains may also be developed independently of commercial suppliers, according to protocols well-known to the skilled person. Such protocols are e.g. described in “Monoclonal Antibody Production” (National Academy Press, Washington, DC, USA, 1999), Carey-Hanly et al. (ILAR Journal, Volume 37, Issue 3, 1995, Pages 93-118), Ilgu and Nilsen-Hamilton (Analyst. 2016 Mar. 7; 141(5): 1551-1568). Reagents may also comprise antibody derivates or fragments, such as Fab, Fab′, F(ab′)2, Fv fragments; diabodies; single-domain antibodies (sdAb, Desmyter et al. (1996) Nat. Structure Biol. 3:803-811), nanobodies, single-chain Fv (scFv, Huston et al. (1988) Proc. Natl. Acad. Sci. USA 85, 5879-5883), divalent scFV (di-scFvs), tandem scFvs, triabodies, diabodies, single-chain diabodies (scDb), bi-specific T-cell engagers (BiTEs, Kufer et al. (2004) Trends Biotechnol. 22:238-244), and Dual Affinity Retargeting molecules (DARTs, diabodies additionally stabilized through a C-terminal disulfide bridge). The specificity of analyte-specific reagents with regard to the intended detection assay may be evaluated using the framework proposed by the International Working Group for Antibody Validation, cited above.

Further, the analyte-specific binding domain may bind to the analyte directly or indirectly. In other words, the detection probe may be a primary reagent which binds directly to the analyte, or a secondary reagent which binds indirectly, by virtue of binding to an intermediate molecule (a primary reagent) which is itself bound directly to the analyte.

In addition to the analyte-specific binding domain, a detection probe as used in the present invention also comprises a nucleic acid moiety, also referred to herein as an oligonucleotide. The oligonucleotide must be long enough to comprise the necessary functional elements used in the detection assay for which the detection probe is intended to be used. That is, at least a sequence capable of generating an identification sequence in the reporter molecule. This is typically 5-20 nucleotides, such as 5-10, 5-15, 10-15 or 15-20 nucleotides. The oligonucleotide may also contain sequences related to primer sites and/or sequencing adaptors for read-out, as known in the art. Generally, the oligonucleotide has a length in the range of 20-100 nucleotides, but may be shorter or longer as required in the specific detection assay in which the detection probe is intended to be used.

Conjugation of a nucleic acid moiety to an antibody can be performed in several ways known to the skilled person, e.g. as reviewed by Dugal-Tessier et al. (J. Clin. Med. 2021, 10,838). Commercial kits for preparing antibody-oligonucleotide conjugates are also readily available from a number of suppliers. The oligonucleotides may be coupled to the analyte binding domains by any means known in the art, and which may be desired or convenient and may be direct, or indirect, e.g. via a linking group. For example, the domains may be associated with one another by covalent linkage (e.g. chemical cross-linking) or by non-covalent association e.g. via streptavidin-biotin based coupling (biotin being provided on one domain, particularly the oligonucleotide domain, and streptavidin on the other).

The oligonucleotide and analyte binding domain are joined together either directly through a bond or indirectly through a linking group. Where linking groups are employed, such groups may be chosen to provide for covalent attachment of the nucleic acid moiety and analyte binding domain through the linking group. The linking group, when present, is in many embodiments biologically inert. In representative embodiments, the linking group is generally at least about 50 Daltons, usually at least about 100 Daltons and may be as large as 1000 Daltons or larger, for example, up to 1000000 Daltons if the linking group contains a spacer, but generally will not exceed about 500 Daltons and usually will not exceed about 300 Daltons. Generally, such linkers will comprise a spacer group terminated at either end with a reactive functionality capable of covalently bonding to the nucleic acid domain or analyte binding domain. Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4 -diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine.

Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides.

Specific linker groups that may find use in the subject proximity probes include heterofunctional compounds, such as azidobenzoyl hydrazide, N-[4-(p-azidosalicylamino)butyl]-3′-[2′-pyridyldithio]propionamide, bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl[4-azidophenyl]-1,3′-dithiopropionate, N-succinimidyl [4-iodoacetyl]aminobenzoate, glutaraldehyde, and succinimidyl-4-[N-maleimidomethyl]cyclohexane-1-carboxylate, 3-(2-pyridyldithio)propionic acid N-hydroxysuccinimide ester (SPDP), 4-(Nmaleimidomethyl)-cyclohexane-1-carboxylic acid N-hydroxysuccinimide ester (SMCC), and the like.

The nucleic acid domain of the detection probes may be made up of ribonucleotides and/or deoxyribonucleotides as well as synthetic nucleotide residues that are capable of participating in Watson-Crick type or analogous base pair interactions. Thus, the nucleic acid domain may be DNA or RNA or a combination or any modification thereof e.g. PNA or other derivatives containing non-nucleotide backbones.

In one embodiment, the detection probes are manufactured by coupling a universal oligonucleotide to the analyte-specific binding domain and subsequently hybridizing a tag oligonucleotide to the universal oligonucleotide, wherein the tag oligonucleotide comprises a sequence capable of generating the identification sequence in a reporter molecule, a sequence complementary to the universal oligonucleotide to facilitate hybridization, and any other functional sequences necessary to perform the detection assay for which the detection probes are intended. Methods for manufacturing such detection probes are described i.a. in international patent publication WO2017/068116.

Where proximity probes are used as the detection probes, each nucleic acid moiety of a proximity probe of the matched pair or more may comprise an identification sequence, or a partial identification sequence. The reporter molecule which is generated may comprise an identification sequence from each of the matched proximity probes. In other words, the identification sequence of the reporter molecule may be a combination, or composite, of the identification sequences of the individual nucleic acid moieties of matched proximity probes. The identification sequences of individual matched proximity probes may be the same or different. In an embodiment, each identification sequence in matched proximity probes is indicative of, or corresponds to, the analyte of interest. However, it is not required for a particular identification sequence of an individual proximity probe to be indicative of an analyte of interest-it is the identification sequence of the reporter nucleic acid molecule which is indicative of the analyte of interest. As indicated above, the reporter identification sequence may be a combination or composite. Alternatively, the identification sequence of the reporter nucleic acid molecule may be derived from the nucleic acid moiety of a single proximity probe (although it will be understood that interaction of the nucleic acid moieties of matched proximity probes will be required for the reporter nucleic acid molecule to form).

In an embodiment, the analyte-specific binding domain is not a nucleic acid which binds by hybridization. In another embodiment, the analyte-specific binding domain is not a nucleic acid. In another embodiment, the detection probe is not composed wholly of nucleic acid. In another embodiment, the detection probe is not a gene-specific probe. In another embodiment, the detection probe is not a padlock probe. In another embodiment, the detection probes are not for use in a microscopy-based optical detection method.

As noted above, the detection probes within a set comprise detection probes specific for (or directed to) the same analyte of interest. In use, it is not required that each detection probe in a set is used for the detection of that analyte. Accordingly, in an embodiment, the detection probes within a set are not designed or intended for use together in a method of detection of an analyte.

In one embodiment of the library according to the invention, each detection probe comprises a matched pair of proximity probes, each proximity probe comprising an analyte-specific binding domain and a nucleic acid moiety, both analyte-specific binding domains of each matched pair of proximity probes being capable of binding specifically to the same analyte of interest, and the nucleic acid moieties of a matched pair of proximity probes being capable of together forming a reporter nucleic acid molecule comprising an identification sequence identifying the respective analyte of interest.

This embodiment is particularly relevant for implementing the present invention in proximity assays, such as Proximity Extension Assays and Proximity Ligation Assays.

As well as the proximity assays developed by Olink Proteomics AB and others as mentioned and referenced above, the libraries of the invention may find application in various commercial assays, available in the art or in development.

In one embodiment, each set of detection probes comprises five or less detection probes. Each set of detection probes may independently comprise one, two, three, four or five detection probes. In some embodiments, all sets of detection probes have the same number of members. In some embodiments, all sets of detection probes have two members. In some embodiments, all sets of detection probes have three members. In some embodiments, all sets of detection probes have four members. In some embodiments, all sets of detection probes have five members. In a presently preferred embodiment, all sets of detection probes have three members.

In some embodiments, the library comprises a set of two or more detection probes for at least 25% of the analytes of interest. In some embodiments, the library comprises a set of two or more detection probes for at least 50%, 60%, 70%, 75%, 80%, 85%, 90%, 95%, 96%, 97%, 98%, 99% or 100% of the analytes of interest.

In some embodiments, the number of unique identification sequences in the library is less than the number of members of the set of analytes of interest.

In some embodiments, the library is adapted for selecting an n-plex panel of detection probes, the adaptation comprising including 1.5n-10n unique identification sequences in the library. That is, the library is set up so that a user can select n analytes of interest, wherein n is any integer, and compose a panel consisting of n detection probes, each specific to one analyte. The library is adapted to this by including enough unique identification sequences in the library to ensure that as high percentage as possible of possible panels can actually be compiled, while at the same time not including too many unique identification sequences, in order to keep manufacturing costs down. It has been found that including 1.5n-3n unique identification sequences in the library is a reasonable trade-off between these two requirements, but under certain circumstances it may be preferable to include more unique identification sequences, such as up to 5n, 7n or 10n.

It follows that a library adapted for selecting panels of a certain plexgrade n is also suitable for selecting panels of slightly lower and higher plexgrade. For instance, a library adapted for selecting a 21-plex panel is also well-suited for selecting a 15-plex panel and a 24-plex panel. In some embodiments, n is an integer between 1 and 100.

One advantage of the invention is that readout of assay results can be standardized across a number of assay panels and identification sequences can be re-used to correlate to different analytes of interest in differently composed panels. In the readout step, the amount of reporter nucleic acid molecules carrying a certain identification sequence is quantified and assigned to its corresponding analyte of interest for the specific panel of assays that is being read.

As is known in the art, different detection modalities for the readout are possible, These include sequencing. Thus, for example, an identification sequence may be a barcode which is sequenced. Any method of sequencing may be used, including sequencing-by-synthesis and sequencing-by-hybridization methods. Thus, depending on the nature of the ID sequence, any suitable method may be used to identify the ID sequence, and this may involve the use of hybridization probes and/or primers. For example, the detection (readout) step may involve amplifying the reporter nucleic acid using one or more primers, at least one of which binds to the ID sequence. Alternatively, the detection method may involve amplifying the reporter and detecting the amplicons by means of specific hybridization probes which bind to the ID sequence (or to a complement thereof). In sequencing-by-hybridization, barcodes can be decoded using labelled hybridization probes, including in combinatorial fashion.

Sequencing advantageously allows a high level of multiplexing and is a convenient method of detection. As noted above, any form of sequencing may be used, including any method of sequencing-by-synthesis, for example, pyrosequencing, reversible dye terminator sequencing and ion torrent sequencing. Particularly, high throughput methods of sequencing are used, and especially massively parallel DNA sequencing. Massively parallel DNA sequencing using the reversible dye terminator method may be performed, for instance, using an Illumina® NovaSeq™ system.

In another embodiment, the ID sequences are primer binding sites, e.g. for a PCR primer, although other amplification methods may be used.

In still further embodiments, the ID sequences may be restriction sites (i.e. a nucleotide sequence recognized by a restriction enzyme). In this embodiment, the nucleic acid domain of a proximity probe may comprise a different restriction site (such that it is recognized and cleaved by a different restriction enzyme). Different combinations of restriction enzymes may thus be applied to differentiate different reporter nucleic acids.

Amplification methods based on PCR are convenient and conveniently the readout may involve quantitative PCR (qPCR) or real-time PCR. The amplicons may be detected using any convenient protocol, including the use of dyes and stains, or labels, e.g. intercalating dyes, or labelled probes which bind to the amplicons. These include molecular beacons and such like, e.g. probes with FRET labels etc.

For instance, when readout is performed by qPCR, it is preferable to be able to provide a limited set of qPCR primers that work for all panels of assays, regardless of the assay content of the various panels. It is also preferable to keep the number of qPCR primers low to reduce cost, risk of mismatched binding and other biological artefacts. With the present invention, it is possible to select a set of detection probes where all detection probes generate reporter molecules with unique identification sequences, while at the same time all those identification sequences also correspond to a limited set of qPCR primer binding sites, so that a corresponding limited set of qPCR primers can be used for read-out of the panel. In this way, the same set of qPCR primers can be used for readout of any panel compiled from the library according to the invention.

Thus, in one embodiment, the unique identification sequences in the library can be made to correspond to a set of qPCR primers that can be used for readout of any panel compiled from the library according to the invention.

In one embodiment, the identification sequences are binding sites for qPCR primers.

In one embodiment, the identification sequences are barcode sequences.

In one embodiment, the set of analytes of interest comprises 100-30,000 analytes of interest.

In a further aspect, the present invention relates to a method for composing a panel of detection probes for a group of analytes of interest, said group of analytes of interest being a subset of a larger set of analytes of interest, said method comprising selecting, from a library according to the invention, one detection probe per analyte in the group of analytes for inclusion in the panel so that each selected detection probe is capable of generating a reporter nucleic acid molecule having an identification sequence that is unique within the composed panel.

In some embodiments, the detection probes comprise matched pairs of proximity probes.

Information of which unique identification sequence represents which analyte of interest needs to be taken into account in readout of the panel. This information is thus preferably documented when selecting the panel and transferred along to the user and implemented in the readout of the specific panel.

In a further aspect, the present invention relates to a computer program comprising instructions causing a computer to perform the method according to the invention.

Performance of the assays using panels composed from the libraries according to the present invention may be done as is previously known in the art, as described i.a. in Assarsson et al., PLOS 1, 2014, 9, 4, e95192; Lundberg et al., Molecular & Cellular Proteomics 10:10.1074/mcp.M110.004978, 1-10, 2011; and Wik et al., 2021, Mol Cell Proteomics 20, 100168.

The invention will be further described in the following illustrative example(s). The example(s) are merely for facilitating understanding of the invention and shall not be construed as limiting the scope of the invention, which is that of the appended claims.

All prior publications cited in the present specification are incorporated by reference in their entirety.

### EXAMPLES

A set of two hundred (200) human proteins were chosen as analytes of interest based on their general relevance to biological pathways related to inflammation. It was decided that the available plexgrade should be 21, i.e. any selected panel should be composed of up to 21 detection probes.

Matched pairs of proximity probes as commonly used in PEA were used as detection probes, and qPCR primer binding sites were used as identification sequences. For each analyte of interest, a set of three detection probes was prepared, each having a unique identification sequence. For the entire library, forty-five unique identification sequences were used, correlating to the qPCR primers comprised in the qPCR readout primers for Olink® Target 48 (Olink Proteomics AB).

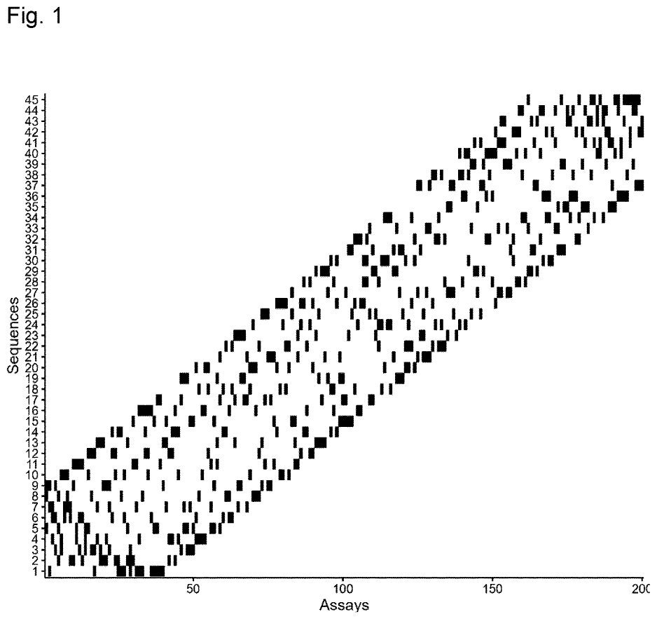

Each set of detection probes was assigned a unique set of three separate identification sequences. That is, no two sets of detection probes had the same combination of identification sequences and no two members of the same set had the same identification sequence. The distribution of identification sequences over the detection probe sets is visualized in FIG. 1.

A simulation was run to determine how many of all possible 21-plex panels could be produced from the library. It was found that the fulfilment rate was 99.9%, i.e. only 0.1% of possible panels cannot be delivered using the library according to the example.

1. A library, comprising:
a plurality of detection probes suitable for detection of a set of analytes of interest,
each detection probe comprising an analyte-specific binding domain and a nucleic acid moiety, the nucleic acid moiety of a detection probe being capable of generating a reporter nucleic acid molecule comprising an identification sequence identifying the respective analyte of interest,
said library being characterized in that the library comprises, for one or more analytes of interest, a set of two or more detection probes wherein each detection probe within the set is capable of generating a reporter nucleic acid molecule comprising an identification sequence that differ from the identification sequences generated by the other members of the same set.

2. The library according to claim 1, wherein each detection probe comprises a matched pair of proximity probes, each proximity probe comprising an analyte-specific binding domain and a nucleic acid moiety, both analyte-specific binding domains of each matched pair of proximity probes being capable of binding specifically to the same analyte of interest, and the nucleic acid moieties of a matched pair of proximity probes being capable of together forming a reporter nucleic acid molecule comprising an identification sequence identifying the respective analyte of interest.

3. The library according to claim 1 wherein each set of detection probes comprises five or less detection probes.

4. The library according to claim 1, wherein the library comprises a set of two or more detection probes for at least 25% of the analytes of interest.

5. The library according to claim 1, wherein the number of unique identification sequences in the library is less than the number of members of the set of analytes of interest.

6. The library according to claim 1, wherein the library is adapted for selecting an n-plex panel of detection probes, the adaptation comprising including 1.5n-10n unique identification sequences in the library.

7. The library according to claim 6, wherein n is an integer between 1 and 100.

8. The library according to claim 1, wherein the identification sequences are binding sites for qPCR primers.

9. The library according to claim 1, wherein the identification sequences are barcode sequences.

10. The library according to claim 1, wherein the set of analytes of interest comprises 100-30,000 analytes of interest.

11. A method for composing a panel of detection probes for a group of analytes of interest, said group of analytes of interest being a subset of a larger set of analytes of interest, said method comprising:
selecting, from a library according to claim 1, one detection probe per analyte in the group of analytes for inclusion in the panel so that each selected detection probe is capable of generating a reporter nucleic acid molecule having an identification sequence that is unique within the composed panel.

12. The method according to claim 11, wherein the detection probes comprise matched pairs of proximity probes.

13. A non-transitory computer-readable medium comprising a set of computer-executable instructions that, when executed by one or more processors, cause:
selecting, from a library according to claim 1, one detection probe per analyte in the group of analytes for inclusion in the panel so that each selected detection probe is capable of generating a reporter nucleic acid molecule having an identification sequence that is unique within the composed panel.

14. The library according to claim 1, wherein the library comprises a set of two or more detection probes for at least 50% of the analytes of interest.

15. The library according to claim 14, wherein the library comprises a set of two or more detection probes for at least 90% of the analytes of interest.

16. The library according to claim 6, the adaptation comprising including 1.5n-3n unique identification sequences in the library.

17. The library according to claim 6, the adaptation comprising including 1.5n-7n unique identification sequences in the library.

18. The library according to claim 1, wherein each set of detection probes independently comprises one, two, three, four or five detection probes.

19. The library according to claim 1, wherein all sets of detection probes have the same number of members.

20. The library according to claim 1, wherein all sets of detection probes have two members, three members, four members, or five members.
